(19) United States
(12) Patent Application Publication
(10) Pub. No.: US 2016/0338638 A1
(43) Pub. Date: Nov. 24, 2016

(54) METHOD AND APPARATUS FOR EVALUATING EXERCISE CAPABILITY USING HEART RATE

(71) Applicant: SAMSUNG ELECTRONICS CO., LTD., Suwon-si (KR)

(72) Inventors: Dae-Geun JANG, Yongin-si (KR); Byunghoon KO, Hwaseong-si (KR); SangKon BAE, Seongnam-si (KR)

(73) Assignee: SAMSUNG ELECTRONICS CO., LTD., Suwon-si (KR)

(21) Appl. No.: 15/156,854

(22) Filed: May 17, 2016

(30) Foreign Application Priority Data
May 20, 2015 (KR) 10-2015-0070560

(51) Int. Cl. A61B 5/00; A61B 5/024; A63B 24/00

(52) U.S. Cl. CPC A61B 5/486; A63B 24/0062; A63B 24/0075; A61B 5/024; A61B 5/7275; A61B 5/4866; A61B 5/4884; A63B 2024/0065; A61B 2503/10

(57) ABSTRACT

A method of evaluating an exercise capability includes measuring a heart rate of a user, generating, based on the measured heart rate, a linearly fitted line and either a quadratically fitted line or a cubically fitted line, detecting a feature parameter using the linearly fitted line and either the quadratic fitted line or the cubically fitted line, and evaluating an exercise capability of the user using either one or both of the feature parameter and physical information of the user.

### CROSS-REFERENCE TO RELATED APPLICATION(S)

This application claims the benefit under 35 USC 119(a) of Korean Patent Application No. 10-2015-0070560 filed on May 20, 2015, in the Korean Intellectual Property Office, the entire disclosure of which is incorporated herein by reference for all purposes.

### BACKGROUND

1. Field

The following description relates to a method and an apparatus for evaluating an exercise capability using a heart rate.

2. Description of Related Art

A general method of evaluating cardiorespiratory fitness may use various measuring devices including a gas analyzer and a blood glucose meter. However, general people lacking technical expertise in handling the devices and medical knowledge for conducting exercise stress tests may be unable to readily use such devices. Thus, various methods have been provided to conveniently measure a physical fitness level or an exercise capability in daily life.

In case of using a heart rate, which is a physiological characteristic, a physical fitness level and an exercise capability may be evaluated under an assumption of a linear relationship between a heart rate and an exercise intensity. However, there may be a nonlinear relationship between an increase in the exercise intensity and a metabolic demand. In such a case, when the exercise intensity increases, a large error may occur in a metabolic characteristic to be estimated using a heart rate. Although various non-metabolic indices including, for example, an age, a gender, and a weight, may be used to supplement the nonlinear relationship, a physiological basis and accuracy in measurement results may still be limited because the indices are not information directly indicating metabolic characteristics of a user.

### SUMMARY

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

In one general aspect, a method of evaluating an exercise capability includes measuring a heart rate of a user; generating, based on the measured heart rate, a linearly fitted line and either a quadratically fitted line or a cubically fitted line; detecting a feature parameter using the linearly fitted line and either the quadratically fitted line or the cubically fitted line; and evaluating an exercise capability of the user using either one or both of the feature parameter and physical information of the user.

The generating of the fitted lines may include generating the linearly fitted line and the quadratically fitted line; and the detecting of the feature parameter may include detecting, as the feature parameter, any one or any combination of any two or more of a first intersection point between the linearly fitted line and the quadratically fitted line; a second intersection point between the linearly fitted line and the quadratically fitted line; a midpoint between the first intersection point and the second intersection point; and a point at which a difference between the linearly fitted line and the quadratically fitted line is largest in a section between the first intersection point and the second intersection point.

The generating of the fitted lines may include generating the linearly fitted line and the cubically fitted line; and the detecting of the feature parameter may include detecting, as the feature parameter, any one or any combination of any two or more of a first intersection point between the linearly fitted line and the cubically fitted line; a second intersection point between the linearly fitted line and the cubically fitted line; a third intersection point between the linearly fitted line and the cubically fitted line; a midpoint between the first intersection point and the second intersection point; a midpoint between the second intersection point and the third intersection point; a point at which a difference between the linearly fitted line and the cubically fitted line is largest in a section between the first intersection point and the second intersection point; and a point at which a difference between the linearly fitted line and the cubically fitted line is largest in a section between the second intersection point and the third intersection point.

The generating of the fitted lines may include generating the fitted lines using any one or any combination of any two or more of a linear interpolation, a polynomial interpolation, a Lagrange interpolation, a bilinear interpolation, a Hermite interpolation, a cubic spline interpolation, an exponential interpolation, a least squares method, a locally weighted least squares method, and a nearest neighbor method.

The evaluating of the exercise capability of the user may include estimating any one or any combination of any two or more of a maximum oxygen consumption, a ventilatory threshold, a lactate threshold, a heart rate deflection point, and a maximal heart rate using either one or both of the feature parameter and the physical information of the user; and evaluating the exercise capability of the user based on a result of the estimating.

The physical information of the user may include any one or any combination of any two or more of a gender, an age, a height, a weight, and a body mass index (BMI) of the user.

The measuring of the heart rate may include measuring a heart rate detected from the user during a gradual exercise or a general activity having an increasing exercise intensity.

In another general aspect, the method may further include selecting, from prestored exercise programs, an exercise program suitable for the exercise capability of the user; receiving, as feedback, a result of performing an exercise from the user performing the exercise according to the selected exercise program; and adjusting the exercise program based on the feedback.

The method may further include comparing the exercise capability of the user to a standard exercise capability based on the physical information of the user; and providing exercise information suitable for a physical fitness level of the user based on a result of the comparing.

The method may further include predicting a metabolic syndrome risk of the user based on the exercise capability of the user; and warning the user of the metabolic syndrome risk.

The predicting of the metabolic syndrome risk may include calculating a health score of the user based on the exercise capability of the user; and estimating a mortality risk based on the health score of the user.

The method may further include providing the user with a lifestyle-related prescription to reduce the metabolic syndrome risk.

In another general aspect, a non-transitory computer-readable storage medium stores instructions to cause computing hardware to perform the method described above.

In another general aspect, an apparatus for evaluating an exercise capability includes a measurer configured to measure a heart rate of a user; a fitted line generator configured to generate, based on the measured heart rate, a linearly fitted line and either a quadratically fitted line or a cubically fitted line; a detector configured to detect a feature parameter using the linearly fitted line and either the quadratically fitted line or the cubically fitted line; and an evaluator configured to evaluate an exercise capability of the user using either one or both of the feature parameter and physical information of the user.

The detector may be further configured to generate the linearly fitted line and the quadratically fitted line, and detect, as the feature parameter, any one or any combination of any two or more of a first intersection point between the linearly fitted line and the quadratically fitted line; a second intersection point between the linearly fitted line and the quadratically fitted line; a midpoint between the first intersection point and the second intersection point; and a point at which a difference between the linearly fitted line and the quadratically fitted line is largest in a section between the first intersection point and the second intersection point.

The detector may be further configured to generate the linearly fitted line and the cubically fitted line, and detect, as the feature parameter, any one or any combination of any two or more of a first intersection point between the linearly fitted line and the cubically fitted line; a second intersection point between the linearly fitted line and the cubically fitted line; a third intersection point between the linearly fitted line and the cubically fitted line; a midpoint between the first intersection point and the second intersection point; a midpoint between the second intersection point and the third intersection point; a point at which a difference between the linearly fitted line and the cubically fitted line is largest in a section between the first intersection point and the second intersection point; and a point at which a difference between the linearly fitted line and the cubically fitted line is largest in a section between the second intersection point and the third intersection point.

The fitted line generator may be further configured to generate the fitted lines using any one or any combination of any two or more of a linear interpolation, a polynomial interpolation, a Lagrange interpolation, a bilinear interpolation, a Hermite interpolation, a cubic spline interpolation, an exponential interpolation, a least squares method, a locally weighted least squares method, and a nearest neighbor method.

The evaluator may be further configured to estimate any one or any combination of any two or more of a maximum oxygen consumption, a ventilatory threshold, a lactate threshold, a heart rate deflection point, and a maximal heart rate using either one or both of the feature parameter and the physical information of the user, and evaluate the exercise capability of the user based on a result of the estimating.

The measurer may be further configured to measure a heart rate detected from the user during a gradual exercise or a general activity having an increasing exercise intensity.

The apparatus may further include either one or both of a warner configured to predict a metabolic syndrome risk of the user based on the exercise capability of the user and warn the user of the metabolic syndrome risk; and a provider configured to provide the user with a lifestyle-related prescription to reduce the metabolic syndrome risk.

Other features and aspects will be apparent from the following detailed description, the drawings, and the claims.

### BRIEF DESCRIPTION OF THE DRAWINGS

FIG. 1 is a graph illustrating an example of a relationship between a heart rate and an oxygen consumption, and an example of a relationship between the heart rate and a lactate concentration.

FIG. 2 is a flowchart illustrating an example of a method of evaluating an exercise capability.

FIG. 3 is a flowchart illustrating another example of a method of evaluating an exercise capability.

FIG. 4 is a flowchart illustrating another example of a method of evaluating an exercise capability.

FIGS. 5A and 5B are graphs illustrating examples of feature parameters to be detected using a linearly fitted line and a quadratically fitted line.

FIG. 6 is a graph illustrating an example of feature parameters to be detected using a linearly fitted line and a cubically fitted line.

FIG. 7 is a diagram illustrating an example of an apparatus for evaluating an exercise capability.

FIG. 8 is a diagram illustrating another example of an apparatus for evaluating an exercise capability.

FIG. 9 is a diagram illustrating another example of an apparatus for evaluating an exercise capability.

Throughout the drawings and the detailed description, the same reference numerals refer to the same elements. The drawings may not be to scale, and the relative size, proportions, and depiction of elements in the drawings may be exaggerated for clarity, illustration, and convenience.

### DETAILED DESCRIPTION

The following detailed description is provided to assist the reader in gaining a comprehensive understanding of the methods, apparatuses, and/or systems described herein. However, various changes, modifications, and equivalents of the methods, apparatuses, and/or systems described herein will be apparent to one of ordinary skill in the art. The sequences of operations herein are merely examples, and are not limited to those set forth herein, but may be changed as will be apparent to one of ordinary skill in the art, with the exception of operations necessarily occurring in a certain order. Also, descriptions of functions and constructions that are well known to one of ordinary skill in the art may be omitted for increased clarity and conciseness.

The features described herein may be embodied in different forms, and are not to be construed as being limited to the examples described herein. Rather, the examples described herein have been provided so that this disclosure will be thorough and complete, and will convey the full scope of the disclosure to one of ordinary skill in the art.

The terminology used herein is for the purpose of describing particular examples only, and is not to limit the disclosure. As used herein, the singular forms “a,” “an,” and “the” are intended to include the plural forms as well, unless the context clearly indicates otherwise. As used herein, the terms “comprises,” “comprising,” “includes,” “including,” “has,” and “having” specify the presence of stated features, numbers, operations, elements, components, and combinations thereof, but do not preclude the presence or addition of one or more other features, numbers, operations, elements, components, and combinations thereof.

Unless otherwise defined, all terms, including technical and scientific terms, used herein have the same meaning as commonly understood by one of ordinary skill in the art to which this disclosure pertains. Terms, such as those defined in commonly used dictionaries, are to be interpreted as having a meaning that is consistent with their meaning in the context of the relevant art, and are not to be interpreted in an idealized or overly formal sense unless expressly so defined herein.

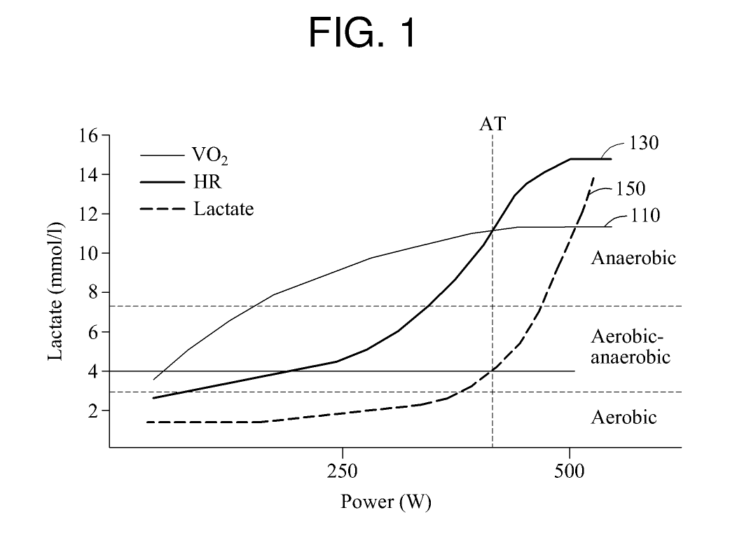

FIG. 1 is a graph illustrating an example of a relationship between a heart rate and an oxygen consumption, and an example of a relationship between the heart rate and a lactate concentration.

Referring to FIG. 1, the graph illustrates a relationship between a heart rate (HR) indicated by a bold line 130 and an oxygen consumption (VO₂) indicated by a solid line 110, and a relationship between the heart rate and a lactate indicated by a dashed line 150.

As illustrated in the graph of FIG. 1, after a lactate concentration reaches 4 millimoles per liter (mmol/l), the lactate concentration increases rapidly. In a relationship between an exercise intensity and a lactate concentration, a point at which the lactate concentration begins rapidly increasing with respect to an increase in the intensity is referred to a lactate threshold. In the graph, the lactate threshold corresponds to an intensity at 4 mmol/l of the lactate concentration, and corresponds to an intensity at an anaerobic threshold (AT).

As illustrated in the graph of FIG. 1, an oxygen consumption continuously increases in proportion to an intensity, and an increase rate of the oxygen consumption decreases rapidly after reaching the anaerobic threshold. A maximum oxygen consumption (VO₂ₘₐₓ) is an oxygen consumption at a point at which the oxygen consumption does not increase further despite an increase in an intensity, after the oxygen consumption increases in proportion to a gradual increase in the intensity. The anaerobic threshold corresponds to the intensity corresponding to the lactate threshold and has a high correlation with the maximum oxygen consumption.

As illustrated in the graph of FIG. 1, a heart rate continuously increases in proportion to an intensity, and an increase rate of a heart rate decreases rapidly after reaching the anaerobic threshold. In a relationship between an intensity and a heart rate at the anaerobic threshold, a heart rate deflection point (HRDP) corresponds to an intensity at which the increase rate of a heart rate begins decreasing rapidly.

Referring to the graph of FIG. 1, a heart rate increases in proportion to an intensity before an arrival at the heart rate deflection point, and has a significant correlation with the maximum oxygen consumption and the lactate threshold that indicate a physical fitness or an exercise capability.

A metabolic index such as the maximum oxygen consumption, the lactate threshold, the heart rate deflection point, a ventilatory threshold, and a maximal heart rate may be used as a standard for measuring a functional ability of a cardiopulmonary system.

In one example, a physical fitness and an exercise capability of a user may be evaluated based on a high correlation between a heart rate and a maximum oxygen consumption, and between a heart rate and a lactate threshold.

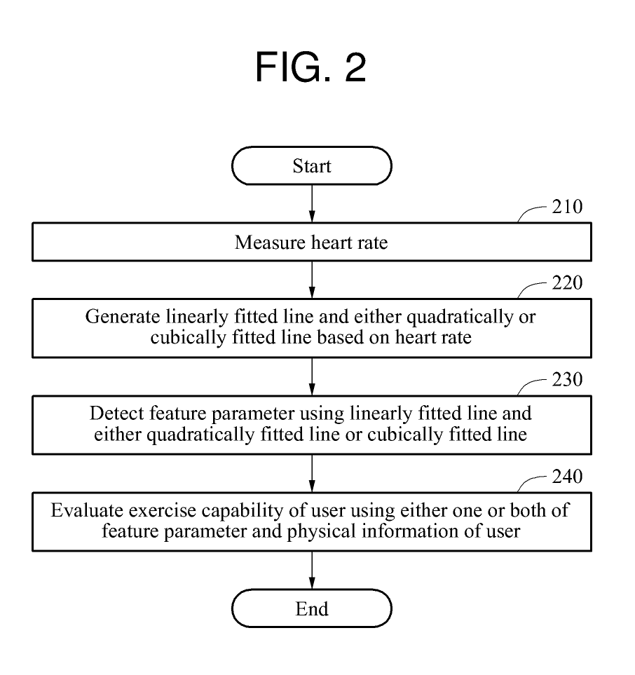

FIG. 2 is a flowchart illustrating an example of a method of evaluating an exercise capability. The method may be performed by an apparatus for evaluating an exercise capability, which will be simply referred to as an exercise capability evaluating apparatus.

Referring to FIG. 2, in operation 210, the exercise capability evaluating apparatus measures a heart rate of a user. The exercise capability evaluating apparatus may measure a heart rate detected from the user while the user performs a gradual exercise such as running on a treadmill, riding a bicycle ergometer, and stepping on a bench step, or a general exercise such as running, jogging, walking, and stair climbing, during which an exercise intensity increases.

The exercise capability evaluating apparatus may be a wearable device including a heart rate sensor or a heart rate meter of various types, for example, a watch type, a bracelet type, a chest type, a patch type, and an in-ear type, or a mobile device connected to a wearable device through wired or wireless communication. The heart rate sensor may include, for example, a photoplethysmogram (PPG) sensor. The exercise capability evaluating apparatus may measure the heart rate of the user using the heart rate sensor or the heart rate meter of various types.

In operation 220, the exercise capability evaluating apparatus generates, based on the measured heart rate, a linearly fitted line and either a quadratically fitted line or a cubically fitted line. The exercise capability evaluating apparatus may generate a fitted line using, for example, a linear interpolation, a polynomial interpolation, a Lagrange interpolation, a bilinear interpolation, a Hermite interpolation, a cubic spline interpolation, an exponential interpolation, a least squares method, a locally weighted least squares method, a nearest neighbor method, or any other method of generating a fitted line known to one of ordinary skill in the art. The interpolation methods described in the foregoing are well known to one of ordinary skill in the art, and thus detailed descriptions of generating a fitted line using such methods will be omitted here. The linearly fitted line and either the quadratic or the cubically fitted line may be used to estimate a heart rate deflection point.

In operation 230, the exercise capability evaluating apparatus detects a feature parameter using the linearly fitted line and either the quadratic or the cubically fitted line. The feature parameter may be a single feature parameter or a plurality of feature parameters. The exercise capability evaluating apparatus detects the feature parameter using the linearly fitted line and the quadratically fitted line, or the linearly fitted line and the cubically fitted line.

In one example, the feature parameter is detected using the linearly fitted line and the quadratically fitted line because a metabolic index, for example, a heart rate, an oxygen consumption, and a lactate concentration in the blood, during an exercise has a nonlinear relationship with an exercise intensity.

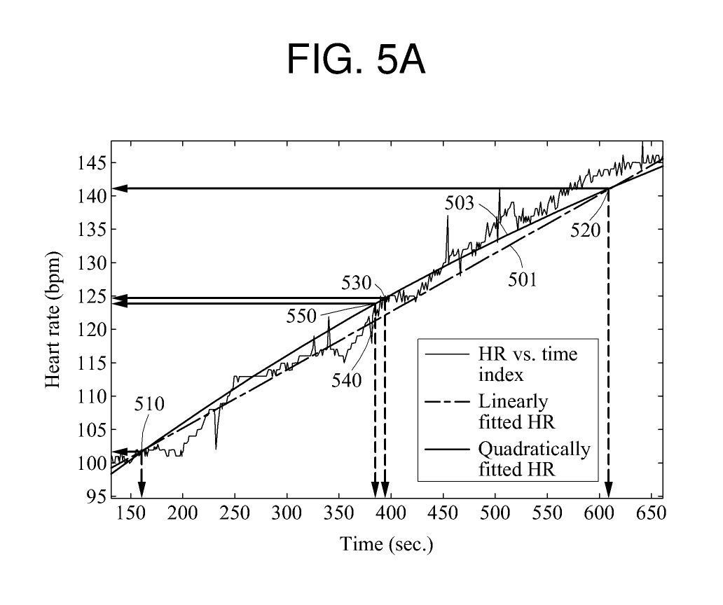

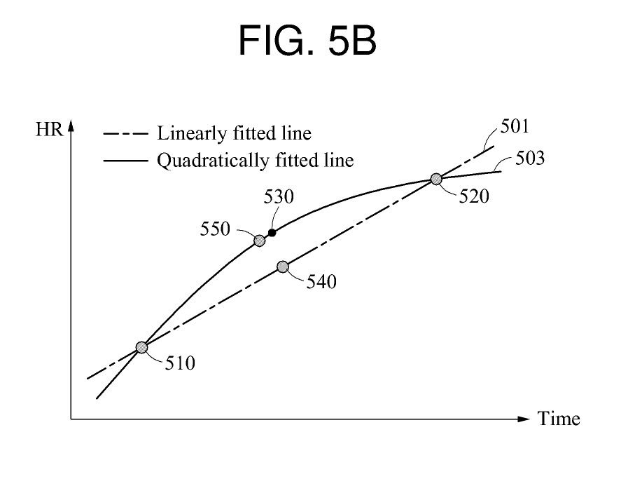

In another example, the feature parameter is detected using the linearly fitted line and the cubically fitted line because a metabolic index during an exercise has a nonlinear relationship with an intensity. For example, a relationship between an intensity and a metabolic index in an exercise program starting from a steady state or a low intensity may be illustrated as in FIGS. 5A and 5B.

By detecting the feature parameter using the fitted lines, the exercise capability evaluating apparatus minimizes an error in evaluation that may occur due to a noise component in a heart rate or a missing heart rate. In one example, the detection of the feature parameter using the linearly fitted line and the quadratically fitted line is useful when a duration of the steady state or the low-intensity exercise state is relatively short, for example, less than or equal to two minutes. In another example, the detection of the feature parameter using the linearly fitted line and the cubically fitted line is useful when the duration of the steady state or the low-intensity exercise state is relatively long, for example, greater than or equal to five minutes.

The described durations of the steady state or the low-intensity exercise state are provided merely as examples, and thus may be modified.

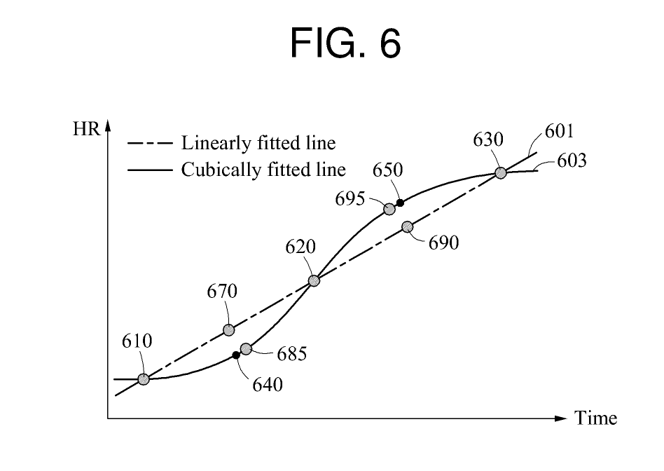

Feature parameters to be detected by the exercise capability evaluating apparatus using the linearly fitted line and the quadratically fitted line will be described with reference to FIGS. 5A and 5B. Feature parameters to be detected by the exercise capability evaluating apparatus using the linearly fitted line and the cubically fitted line will be described with reference to FIG. 6.

In operation 240, the exercise capability evaluating apparatus evaluates an exercise capability of the user using either one or both of the feature parameter detected in operation 230 and physical information of the user. The physical information of the user may be inputted directly from the user, or may be a prestored value. In addition, the physical information of the user may be updated. The physical information of the user may include, for example, a gender, an age, a height, a weight, and a body mass index (BMI) of the user. The BMI is a value obtained by dividing a weight (kg) of the user by of the square of a height of the user (m²).

Based on the exercise capability evaluated in operation 240, the exercise capability evaluating apparatus may provide a personalized exercise service suitable for the user, or provide a method of predicting a metabolic syndrome risk and managing the metabolic syndrome risk.

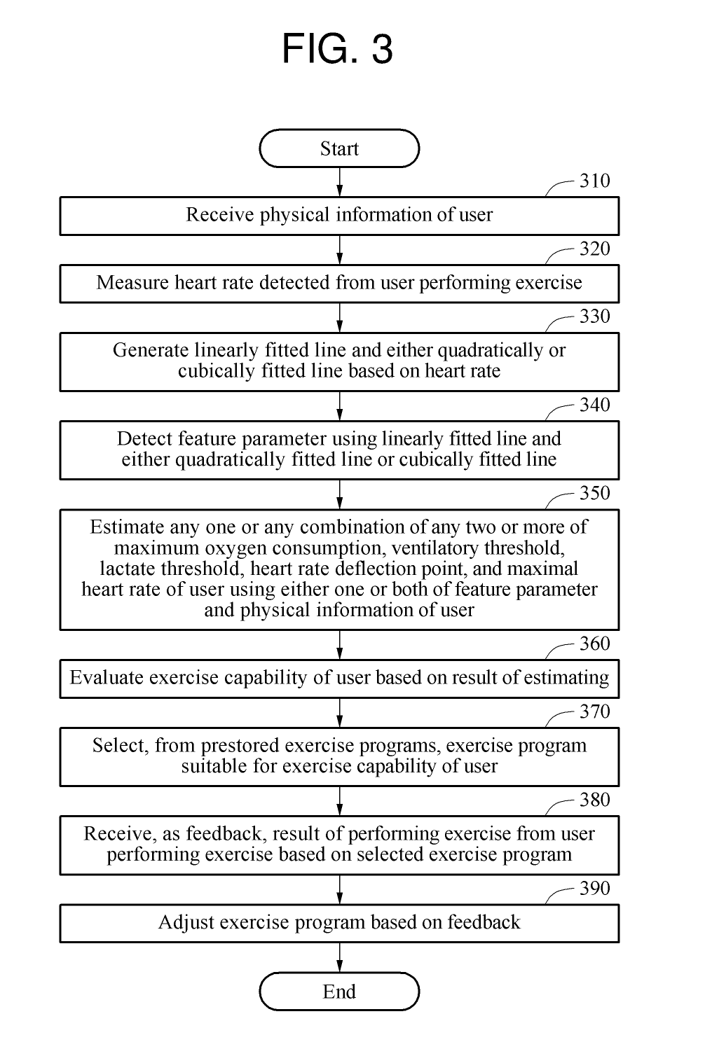

FIG. 3 is a flowchart illustrating another example of a method of evaluating an exercise capability.

Referring to FIG. 3, in operation 310, an exercise capability evaluating apparatus receives physical information of a user. For example, the physical information of the user may include a gender, an age, a height, a weight, and a BMI of the user.

In operation 320, the exercise capability evaluating apparatus measures a heart rate detected from the user while the user performs an exercise having an increasing exercise intensity.

In operation 330, the exercise capability evaluating apparatus generates, based on the heart rate measured in operation 320, a linearly fitted line and either a quadratically fitted line or a cubically fitted line.

In operation 340, the exercise capability evaluating apparatus detects a feature parameter using the linearly fitted line and either the quadratic or the cubically fitted line.

In one example, the exercise capability evaluating apparatus detects, as feature parameters, a first intersection point between the linearly fitted line and the quadratically fitted line, a second intersection point between the linearly fitted line and the quadratically fitted line, a midpoint between the first intersection point and the second intersection point, and a point at which a difference between the linearly fitted line and the quadratically fitted line is largest in a section between the first intersection point and the second intersection point. The midpoint between the first intersection point and the second intersection point is detected on each of the linearly fitted line and the quadratically fitted line. Thus, the exercise capability evaluating apparatus detects a total of five feature parameters using the linearly fitted line and the quadratically fitted line.

In another example, the exercise capability evaluating apparatus detects, as feature parameters, a first intersection point between the linearly fitted line and the cubically fitted line, a second intersection point between the linearly fitted line and the cubically fitted line, a third intersection point between the linearly fitted line and the cubically fitted line, a midpoint between the first intersection point and the second intersection point, a midpoint between the second intersection point and the third intersection point, a point at which a difference between the linearly fitted line and the cubically fitted line is largest in a section between the first intersection point and the second intersection point, and a point at which a difference between the linearly fitted line and the cubically fitted line is largest in a section between the second intersection point and the third intersection point. The midpoint between the first intersection point and the second intersection point and the midpoint between the second intersection point and the third intersection point are detected on each of the linearly fitted line and the cubically fitted line. Thus, the exercise capability evaluating apparatus detects a total of nine feature parameters using the linearly fitted line and the cubically fitted line.

The exercise capability evaluating apparatus minimizes an influence of noise prone to occur during an exercise by detecting the feature parameters using the linearly fitted line and either the quadratic or the cubically fitted line.

In operation 350, the exercise capability evaluating apparatus estimates a metabolic index such as a maximum oxygen consumption, a ventilatory threshold, a lactate threshold, a heart rate deflection point, and a maximal heart rate using either one or both of the feature parameter detected in operation 340 and the physical information of the user.

The exercise capability evaluating apparatus may estimate the maximum oxygen consumption, the ventilatory threshold, the lactate threshold, the heart rate deflection point, and the maximal heart rate of the user using only the feature parameter detected in operation 340, or using both the feature parameter and the physical information of the user.

The exercise capability evaluating apparatus may estimate the metabolic index, such as the maximum oxygen consumption, the ventilatory threshold, the lactate threshold, the heart rate deflection point, and the maximal heart rate of the user, by substituting, in a linear regression equation, the feature parameter and a set of the physical information of the user.

In one example, the exercise capability evaluating apparatus estimates a metabolic index “Y” of the user, for example, the maximum oxygen consumption, the ventilatory threshold, the lactate threshold, the heart rate deflection point, and the maximal heart rate, by substituting a feature parameter, for example, the point detected in operation 340 at which the difference between the linearly fitted line and the quadratically fitted line is largest, in a regression equation 1, for example, Y=α×X+β. A value to be substituted for “X” in the regression equation 1 is a value of a heart rate corresponding to the feature parameter.

In the regression equation 1, in addition to the point at which the difference between the linearly fitted line and the quadratically fitted line is largest, the various feature parameters detected in operation 340 may be used as the feature parameter X. The coefficients “α” and “β” in the regression equation 1 may be different depending on the metabolic index Y to be estimated, and the feature parameter X to be used. For example, the coefficients α and β in the regression equation 1 may change depending on whether the metabolic index Y to be estimated is the maximum oxygen consumption or the maximal heart rate.

In another example, the exercise capability evaluating apparatus estimates a metabolic index “Y” of the user, for example, the maximum oxygen consumption, the ventilatory threshold, the lactate threshold, the heart rate deflection point, or the maximal heart rate of the user, by substituting a feature parameter “X1,” for example, the first intersection point between the linearly fitted line and the cubically fitted line, and one piece of the physical information of the user, for example, a BMI “X2,” in a linear regression equation 2, for example, Y=α1×X1+α2×X2+β. As in the regression equation 1, the coefficients “α1,” “α2,” and “β” in the regression equation 2 may be different depending on the metabolic index Y to be estimated, and the feature parameter X1 and the piece of the physical information X2 to be used.

In another example, the exercise capability evaluating apparatus estimates a metabolic index “Y” of the user, for example, the maximum oxygen consumption, the ventilatory threshold, the lactate threshold, the heart rate deflection point, or the maximal heart rate of the user, by substituting a feature parameter “X1,” for example, the point at which the difference between the linearly fitted line and the quadratically fitted line is the largest, and two pieces of the physical information of the user, for example, an age “X2” and a BMI “X3” of the user, in a regression equation 3, for example, Y=α1×X1+α2×X2+α3×X3+β. The coefficients “α1,” “α2,” “α3,” and “β” in the regression equation 3 may be different depending on the metabolic index Y to be estimated, and the feature parameter X1 and the pieces of the physical information X2 and X3 to be used.

In another example, the exercise capability evaluating apparatus estimates a metabolic index “Y” of the user, for example, the maximum oxygen consumption, the ventilatory threshold, the lactate threshold, the heart rate deflection point, or the maximal heart rate of the user, by substituting a feature parameter “X1,” for example, the second intersection point between the linearly fitted line and the quadratically fitted line, and three pieces of the physical information of the user, for example, an age “X2,” a gender “X3,” and a BMI “X4” of the user, in a regression equation 4, for example, Y=α1×X1+α2×X2+α3×X3+α4×X4+β. The coefficients “α1,” “α2,” “α3,” “α4,” and “β” in the regression equation 4 may be different depending on the metabolic index “Y” to be estimated, the feature parameter X1 and the pieces of information X2, X3, and X4 to be used.

In operation 360, the exercise capability evaluating apparatus evaluates an exercise capability of the user based on a result of the estimating performed in operation 350.

In operation 370, the exercise capability evaluating apparatus selects, from prestored exercise programs, an exercise program suitable for the exercise capability of the user evaluated in operation 360. In addition, the exercise capability evaluating apparatus selects a level of the selected exercise program that is suitable for the evaluated exercise capability of the user. The exercise capability evaluating apparatus provides the user with the selected level or the selected exercise program.

In operation 380, the exercise capability evaluating apparatus receives, as feedback, a result of performing an exercise from the user performing the exercise according to the selected exercise program. The feedback refers to, for example, a level of difficulty the user experiences as a result of performing the exercise program. For example, the exercise capability evaluating apparatus may ask the user about the level of difficulty the user experienced in performing the exercise program, for example, whether the exercise program was easy, moderate, or hard for the user to perform, and receive a response to the inquiry from the user.

In operation 390, the exercise capability evaluating apparatus adjusts the exercise program based on the feedback received in operation 380. The exercise capability evaluating apparatus may modify the exercise program or adjust a level of the exercise based on the received feedback. Information about the exercise program modified or the exercise level adjusted in operation 390 may be transmitted to operation 370 and used by the exercise capability evaluating apparatus to select an exercise program.

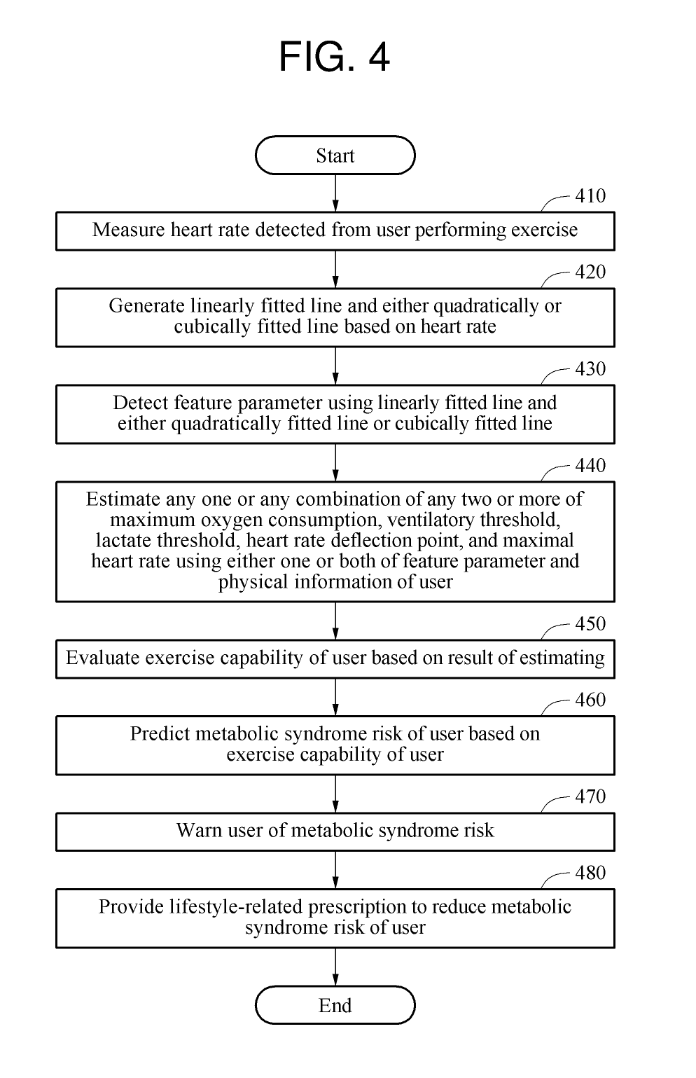

FIG. 4 is a flowchart illustrating another example of a method of evaluating an exercise capability.

Operations 410 through 450 illustrated in FIG. 4 are identical to the operations 320 through 360 illustrated in FIG. 3. Thus, for a detailed description of the operations 410 through 450, reference may be made to the descriptions of the operations 320 through 360 provided with reference to FIG. 3.

Referring to FIG. 4, in operation 460, the exercise capability evaluating apparatus predicts a metabolic syndrome risk of the user based on the exercise capability of the user evaluated in operation 450. The exercise capability evaluating apparatus also calculates a health score of the user based on the exercise capability of the user and estimates a mortality risk of the user based on the calculated health score of the user.

In operation 470, the exercise capability evaluating apparatus warns the user of the metabolic syndrome risk. For example, the exercise capability evaluating apparatus may warn the user of the metabolic syndrome risk using an audio message or by displaying a phrase, for example, “you are currently at risk of developing metabolic syndrome.”

In operation 480, the exercise capability evaluating apparatus provides the user with a lifestyle-related prescription to reduce the metabolic syndrome risk. For example, the lifestyle-related prescription may include an exercise prescription, a nutrition prescription, and any other prescription to reduce the metabolic syndrome risk known to one of ordinary skill in the art.

In addition, the exercise capability evaluating apparatus may compare the exercise capability of the user to a standard exercise capability for a gender and an age of the user based on the physical information of the user, and may provide exercise information suitable for a physical fitness level of the user based on the exercise capability evaluated in operation 450.

FIGS. 5A and 5B are graphs illustrating examples of feature parameters to be detected using a linearly fitted line and a quadratically fitted line.

FIG. 5A is a graph illustrating a linearly fitted line 501 and a quadratically fitted line 503 generated based on a heart rate actually measured and changing over time. FIG. 5B is a simplified graph of the graph illustrated in FIG. 5A.

Referring to the graphs of FIGS. 5A and 5B, an exercise capability evaluating apparatus detects, as feature parameters, a first intersection point 510 at which the linearly fitted line 501 initially meets the quadratically fitted line 503, a second intersection point 520, a point 530 at which a difference between the linearly fitted line 501 and the quadratically fitted line 503 is largest in a section between the first intersection point 510 and the second intersection point 520, a midpoint 540 between the first intersection point 510 and the second intersection point 520 on the linearly fitted line 501, and a midpoint 550 between the first intersection point 510 and the second intersection point 520 on the quadratically fitted line 503.

FIG. 6 is a graph illustrating examples of feature parameters to be detected using a linearly fitted line and a cubically fitted line.

FIG. 6 is a graph illustrating a linearly fitted line 601 and a cubit fitted line 603 generated based on a heart rate measured from a user.

Referring to the graph of FIG. 6, an exercise capability evaluating apparatus detects, as feature parameters, a first intersection point 610, a second intersection point 620, and a third intersection point 630 between the linearly fitted line 601 and the cubically fitted line 603, a point 640 at which a difference between the linearly fitted line 601 and the cubically fitted line 603 is largest in a section between the first intersection point 610 and the second intersection point 620, a point 650 at which a difference between the linearly fitted line 601 and the cubically fitted line 603 is largest in a section between the second intersection point 620 and the third intersection point 630, midpoint 670 between the first intersection point 610 and the second intersection point 620 on the linearly fitted line 601, a midpoint 685 between the first intersection point 610 and the second intersection point 620 on the cubically fitted line 603, midpoint 690 between the second intersection point 620 and the third intersection point 630 on the linearly fitted line 601, and a midpoint 695 between the second intersection point 620 and the third intersection point 630 on the cubically fitted line 603.

The exercise capability evaluating apparatus detects feature parameters using a linearly fitted line and a quadratically fitted line as illustrated in FIGS. 5A and 5B, or using the linearly fitted line and a cubically fitted line as illustrated in FIG. 6, and thus may minimize an error in evaluation that may occur due to a noise component in a heart rate or a missing heart rate.

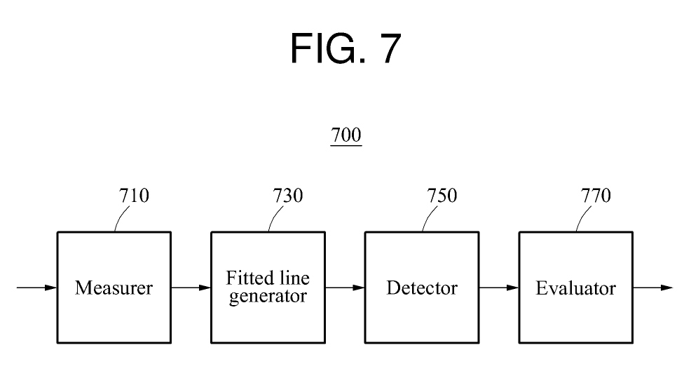

FIG. 7 is a diagram illustrating an example of an exercise capability evaluating apparatus 700.

Referring to FIG. 7, the exercise capability evaluating apparatus 700 includes a measurer 710, a fitted line generator 730, a detector 750, and an evaluator 770.

The measurer 710 measures a heart rate of a user. The measurer 710 measures a heart rate detected from the user while the user performs a gradual exercise or a general activity having an increasing exercise intensity.

The fitted line generator 730 generates, based on the heart rate measured by the measurer 710, a linearly fitted line and either a quadratic or a cubically fitted line. The fitted line generator 730 generates the fitted lines using, for example, a linear interpolation, a polynomial interpolation, a Lagrange interpolation, a bilinear interpolation, a Hermite interpolation, a cubic spline interpolation, an exponential interpolation, a least squares method, a locally weighted least squares method, a nearest neighbor method, or any other method of generating a fitted line known to one of ordinary skill in the art.

The detector 750 detects a feature parameter using the linearly fitted line and either the quadratic or the cubically fitted line generated by the fitted line generator 730.

In one example, the detector 750 detects, as a feature parameter, any one or any combination of any two or more of a first intersection point between the linearly fitted line and the quadratically fitted line, a second intersection line between the linearly fitted line and the quadratically fitted line, a midpoint between the first intersection point and the second intersection point on each of the linearly fitted line and the quadratically fitted line, and a point at which a difference between the linearly fitted line and the quadratically fitted line is largest in a section between the first intersection point and the second intersection point.

In another example, the detector 750 detects, as a feature parameter, any one or any combination of any two or more of a first intersection point between the linearly fitted line and the cubically fitted line, a second intersection point between the linearly fitted line and the cubically fitted line, a third intersection point between the linearly fitted line and the cubically fitted line, a midpoint between the first intersection point and the second intersection point, a midpoint between the second intersection point and the third intersection point, a point at which a difference between the linearly fitted line and the cubically fitted line is largest in a section between the first intersection point and the second intersection point, and a point at which a difference between the linearly fitted line and the cubically fitted line is largest in a section between the second intersection point and the third intersection point.

The evaluator 770 evaluates an exercise capability of the user using either one or both of the feature parameter and physical information of the user. The evaluator 770 estimates any one or any combination of any two or more of a maximum oxygen consumption, a ventilatory threshold, a lactate threshold, a heart rate deflection point, and a maximal heart rate of the user using either one or both of the feature parameter and the physical information of the user. The evaluator 770 evaluates the exercise capability of the user based on a result of the estimating.

The physical information of the user may include, for example, a gender, an age, a height, a weight, and a BMI of the user.

In one example, the evaluator 770 estimates the maximum oxygen consumption of the user based on the feature parameter detected by the detector 750, for example, the first intersection point between the linearly fitted line and the cubically fitted line. In another example, the evaluator 770 estimates the maximal heart rate of the user based on the feature parameter, for example, the point at which the difference between the linearly fitted line and the quadratically fitted line is largest, and the BMI of the user.

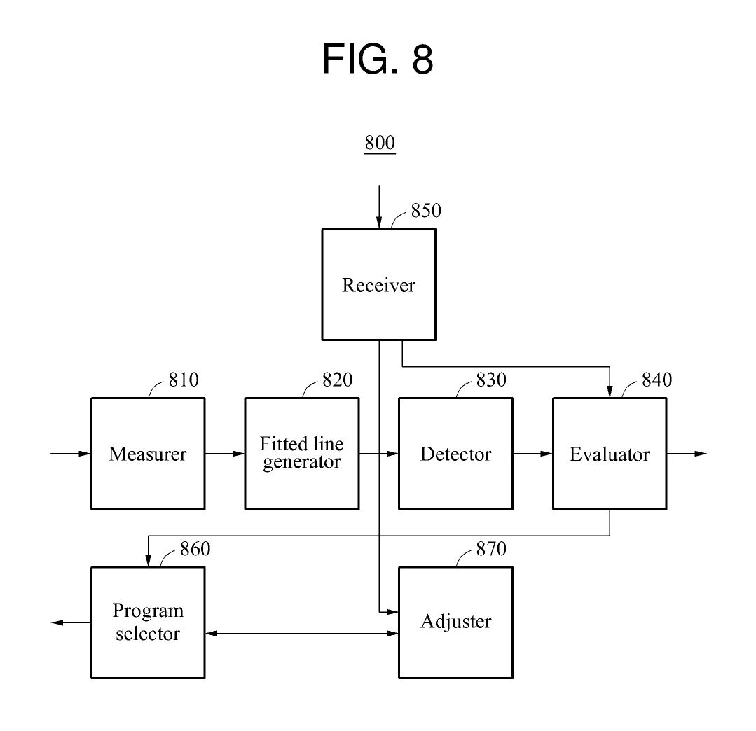

FIG. 8 is a diagram illustrating another example of an exercise capability evaluating apparatus 800.

Referring to FIG. 8, the exercise capability evaluating apparatus 800 includes a measurer 810, a fitted line generator 820, a detector 830, an evaluator 840, a receiver 850, a program selector 860, and an adjuster 870.

For descriptions of the measurer 810, the fitted line generator 820, the detector 830, and the evaluator 840, reference may be made to the descriptions of the measurer 710, the fitted line generator 730, the detector 750, and the evaluator 770 provided with reference to FIG. 7.

The receiver 850 receives, from a user, physical information of the user.

The program selector 860 selects, from prestored exercise programs, an exercise program suitable for the exercise capability of the user evaluated by the evaluator 840.

The receiver 850 receives, as feedback, a result of performing an exercise from the user performing the exercise according to the exercise program selected by the program selector 860.

The adjuster 870 adjusts the exercise program based on the feedback received by the receiver 850.

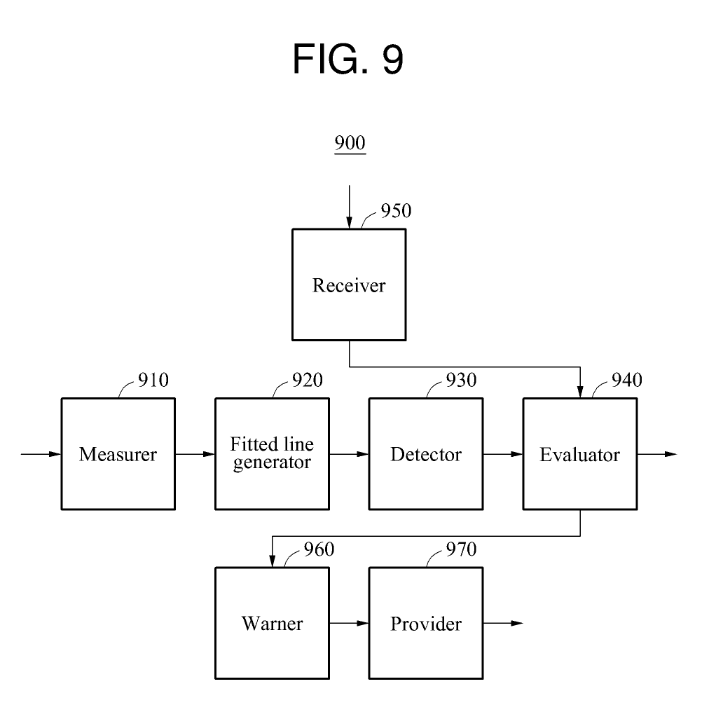

FIG. 9 is a diagram illustrating another example of an exercise capability evaluating apparatus 900.

Referring to FIG. 9, the exercise capability evaluating apparatus 900 includes a measurer 910, a fitted line generator 920, a detector 930, an evaluator 940, a receiver 950, a warner 960, and a provider 970.

For descriptions of the measurer 910, the fitted line generator 920, the detector 930, and the evaluator 940, reference may be made to the descriptions of the measurer 710, the fitted line generator 730, the detector 750, and the evaluator 770 provided with reference to FIG. 7.

The receiver 950 receives, from a user, physical information of the user.

The warner 960 predicts a metabolic syndrome risk of the user based on the exercise capability of the user evaluated by the evaluator 940, and warns the user of the metabolic syndrome risk.

For example, the warner 960 may calculate a health score of the user based on the exercise capability of the user and estimate a mortality risk of dying from a metabolic disease based on the calculated health score of the user, and warn the user of the mortality risk.

The provider 970 provides the user with a lifestyle-related prescription to reduce the metabolic syndrome risk. The lifestyle-related prescription may include advice on a lifestyle or a habit of the user, an exercise prescription, and a nutrition prescription including a diet.

The exercise capability evaluating apparatus 700, the measurer 710, the fitted line generator 730, the detector 750, and the evaluator 770 illustrated in FIG. 7, the exercise capability evaluating apparatus 800, the measurer 810, the fitted line generator 820, the detector 830, the evaluator 840, the receiver 850, the program selector 860, and the adjuster 870 illustrated in FIG. 8, and the exercise capability evaluating apparatus 900, the fitted line generator 920, the detector 930, the evaluator 940, the receiver 950, the warner 960, and the provider 970 illustrated in FIG. 9 that perform the operations described herein with respect to FIGS. 1-9 are implemented by hardware components. Examples of hardware components include controllers, sensors, generators, drivers, memories, comparators, arithmetic logic units, adders, subtractors, multipliers, dividers, integrators, and any other electronic components known to one of ordinary skill in the art. In one example, the hardware components are implemented by computing hardware, for example, by one or more processors or computers. A processor or computer is implemented by one or more processing elements, such as an array of logic gates, a controller and an arithmetic logic unit, a digital signal processor, a microcomputer, a programmable logic controller, a field-programmable gate array, a programmable logic array, a microprocessor, or any other device or combination of devices known to one of ordinary skill in the art that is capable of responding to and executing instructions in a defined manner to achieve a desired result. In one example, a processor or computer includes, or is connected to, one or more memories storing instructions or software that are executed by the processor or computer. Hardware components implemented by a processor or computer execute instructions or software, such as an operating system (OS) and one or more software applications that run on the OS, to perform the operations described herein with respect to FIGS. 1-9. The hardware components also access, manipulate, process, create, and store data in response to execution of the instructions or software. For simplicity, the singular term “processor” or “computer” may be used in the description of the examples described herein, but in other examples multiple processors or computers are used, or a processor or computer includes multiple processing elements, or multiple types of processing elements, or both. In one example, a hardware component includes multiple processors, and in another example, a hardware component includes a processor and a controller. A hardware component has any one or more of different processing configurations, examples of which include a single processor, independent processors, parallel processors, single-instruction single-data (SISD) multiprocessing, single-instruction multiple-data (SIMD) multiprocessing, multiple-instruction single-data (MISD) multiprocessing, and multiple-instruction multiple-data (MIMD) multiprocessing.

The methods illustrated in FIGS. 2-4 that perform the operations described herein with respect to FIGS. 1-9 are performed by a processor or a computer as described above executing instructions or software to perform the operations described herein.

Instructions or software to control a processor or computer to implement the hardware components and perform the methods as described above are written as computer programs, code segments, instructions or any combination thereof, for individually or collectively instructing or configuring the processor or computer to operate as a machine or special-purpose computer to perform the operations performed by the hardware components and the methods as described above. In one example, the instructions or software include machine code that is directly executed by the processor or computer, such as machine code produced by a compiler. In another example, the instructions or software include higher-level code that is executed by the processor or computer using an interpreter. Programmers of ordinary skill in the art can readily write the instructions or software based on the block diagrams and the flow charts illustrated in the drawings and the corresponding descriptions in the specification, which disclose algorithms for performing the operations performed by the hardware components and the methods as described above.

The instructions or software to control a processor or computer to implement the hardware components and perform the methods as described above, and any associated data, data files, and data structures, are recorded, stored, or fixed in or on one or more non-transitory computer-readable storage media. Examples of a non-transitory computer-readable storage medium include read-only memory (ROM), random-access memory (RAM), flash memory, CD-ROMs, CD-Rs, CD+Rs, CD-RWs, CD+RWs, DVD-ROMs, DVD-Rs, DVD+Rs, DVD-RWs, DVD+RWs, DVD-RAMs, BD-ROMs, BD-Rs, BD-R LTHs, BD-REs, magnetic tapes, floppy disks, magneto-optical data storage devices, optical data storage devices, hard disks, solid-state disks, and any device known to one of ordinary skill in the art that is capable of storing the instructions or software and any associated data, data files, and data structures in a non-transitory manner and providing the instructions or software and any associated data, data files, and data structures to a processor or computer so that the processor or computer can execute the instructions. In one example, the instructions or software and any associated data, data files, and data structures are distributed over network-coupled computer systems so that the instructions and software and any associated data, data files, and data structures are stored, accessed, and executed in a distributed fashion by the processor or computer.

While this disclosure includes specific examples, it will be apparent to one of ordinary skill in the art that various changes in form and details may be made in these examples without departing from the spirit and scope of the claims and their equivalents. The examples described herein are to be considered in a descriptive sense only, and not for purposes of limitation. Descriptions of features or aspects in each example are to be considered as being applicable to similar features or aspects in other examples. Suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A method of evaluating an exercise capability comprising:
measuring a heart rate of a user;
generating, based on the measured heart rate, a linearly fitted line and either a quadratically fitted line or a cubically fitted line;
detecting a feature parameter using the linearly fitted line and either the quadratically fitted line or the cubically fitted line; and
evaluating an exercise capability of the user using either one or both of the feature parameter and physical information of the user.

2. The method of claim 1, wherein the generating of the fitted lines comprises generating the linearly fitted line and the quadratically fitted line; and
the detecting of the feature parameter comprises detecting, as the feature parameter, any one or any combination of any two or more of:
a first intersection point between the linearly fitted line and the quadratically fitted line;
a second intersection point between the linearly fitted line and the quadratically fitted line;
a midpoint between the first intersection point and the second intersection point; and
a point at which a difference between the linearly fitted line and the quadratically fitted line is largest in a section between the first intersection point and the second intersection point.

3. The method of claim 1, wherein the generating of the fitted lines comprises generating the linearly fitted line and the cubically fitted line; and
the detecting of the feature parameter comprises detecting, as the feature parameter, any one or any combination of any two or more of:
a first intersection point between the linearly fitted line and the cubically fitted line;
a second intersection point between the linearly fitted line and the cubically fitted line;
a third intersection point between the linearly fitted line and the cubically fitted line;
a midpoint between the first intersection point and the second intersection point;
a midpoint between the second intersection point and the third intersection point;
a point at which a difference between the linearly fitted line and the cubically fitted line is largest in a section between the first intersection point and the second intersection point; and
a point at which a difference between the linearly fitted line and the cubically fitted line is largest in a section between the second intersection point and the third intersection point.

4. The method of claim 1, wherein the generating of the fitted lines comprises generating the fitted lines using any one or any combination of any two or more of a linear interpolation, a polynomial interpolation, a Lagrange interpolation, a bilinear interpolation, a Hermite interpolation, a cubic spline interpolation, an exponential interpolation, a least squares method, a locally weighted least squares method, and a nearest neighbor method.

5. The method of claim 1, wherein the evaluating of the exercise capability of the user comprises:
estimating any one or any combination of any two or more of a maximum oxygen consumption, a ventilatory threshold, a lactate threshold, a heart rate deflection point, and a maximal heart rate using either one or both of the feature parameter and the physical information of the user; and
evaluating the exercise capability of the user based on a result of the estimating.

6. The method of claim 5, wherein the physical information of the user comprises any one or any combination of any two or more of a gender, an age, a height, a weight, and a body mass index (BMI) of the user.

7. The method of claim 1, wherein the measuring of the heart rate comprises measuring a heart rate detected from the user during a gradual exercise or a general activity having an increasing exercise intensity.

8. The method of claim 1, further comprising:
selecting, from prestored exercise programs, an exercise program suitable for the exercise capability of the user;
receiving, as feedback, a result of performing an exercise from the user performing the exercise according to the selected exercise program; and
adjusting the exercise program based on the feedback.

9. The method of claim 1, further comprising:
comparing the exercise capability of the user to a standard exercise capability based on the physical information of the user; and
providing exercise information suitable for a physical fitness level of the user based on a result of the comparing.

10. The method of claim 1, further comprising:
predicting a metabolic syndrome risk of the user based on the exercise capability of the user; and
warning the user of the metabolic syndrome risk.

11. The method of claim 10, wherein the predicting of the metabolic syndrome risk comprises:
calculating a health score of the user based on the exercise capability of the user; and
estimating a mortality risk based on the health score of the user.

12. The method of claim 10, further comprising providing the user with a lifestyle-related prescription to reduce the metabolic syndrome risk.

13. A non-transitory computer-readable storage medium storing instructions to cause computing hardware to perform the method of claim 1.

14. An apparatus for evaluating an exercise capability comprising:
a measurer configured to measure a heart rate of a user;
a fitted line generator configured to generate, based on the measured heart rate, a linearly fitted line and either a quadratically fitted line or a cubically fitted line;
a detector configured to detect a feature parameter using the linearly fitted line and either the quadratically fitted line or the cubically fitted line; and
an evaluator configured to evaluate an exercise capability of the user using either one or both of the feature parameter and physical information of the user.

15. The apparatus of claim 14, wherein the detector is further configured to generate the linearly fitted line and the quadratically fitted line, and detect, as the feature parameter, any one or any combination of any two or more of:
a first intersection point between the linearly fitted line and the quadratically fitted line;
a second intersection point between the linearly fitted line and the quadratically fitted line;
a midpoint between the first intersection point and the second intersection point; and
a point at which a difference between the linearly fitted line and the quadratically fitted line is largest in a section between the first intersection point and the second intersection point.

16. The apparatus of claim 14, wherein the detector is further configured to generate the linearly fitted line and the cubically fitted line, and detect, as the feature parameter, any one or any combination of any two or more of:
a first intersection point between the linearly fitted line and the cubically fitted line;
a second intersection point between the linearly fitted line and the cubically fitted line;
a third intersection point between the linearly fitted line and the cubically fitted line;
a midpoint between the first intersection point and the second intersection point;
a midpoint between the second intersection point and the third intersection point;
a point at which a difference between the linearly fitted line and the cubically fitted line is largest in a section between the first intersection point and the second intersection point; and
a point at which a difference between the linearly fitted line and the cubically fitted line is largest in a section between the second intersection point and the third intersection point.

17. The apparatus of claim 14, wherein the fitted line generator is further configured to generate the fitted lines using any one or any combination of any two or more of a linear interpolation, a polynomial interpolation, a Lagrange interpolation, a bilinear interpolation, a Hermite interpolation, a cubic spline interpolation, an exponential interpolation, a least squares method, a locally weighted least squares method, and a nearest neighbor method.

18. The apparatus of claim 14, wherein the evaluator is further configured to estimate any one or any combination of any two or more of a maximum oxygen consumption, a ventilatory threshold, a lactate threshold, a heart rate deflection point, and a maximal heart rate using either one or both of the feature parameter and the physical information of the user, and evaluate the exercise capability of the user based on a result of the estimating.

19. The apparatus of claim 14, wherein the measurer is further configured to measure a heart rate detected from the user during a gradual exercise or a general activity having an increasing exercise intensity.

20. The apparatus of claim 14, further comprising either one or both of:
a warner configured to predict a metabolic syndrome risk of the user based on the exercise capability of the user and warn the user of the metabolic syndrome risk; and
a provider configured to provide the user with a lifestyle-related prescription to reduce the metabolic syndrome risk.
